(19) United States
(12) Patent Application Publication
(10) Pub. No.: US 2024/0124824 A1
(43) Pub. Date: Apr. 18, 2024

(54) LIGHTING DEVICE AND INCUBATOR FOR MICROALGAE GROWTH EXPERIMENTS

(71) Applicants: David BLANCO RAYÓN, Bilbao (ES); Universidad del País Vasco/EuskalHerriko Unibertsitatea, Leioa (ES)

(72) Inventors: David BLANCO RAYÓN, Bilbao (ES); Esther BLANCO RAYÓN, Leioa (ES)

(21) Appl. No.: 18/398,722

(22) Filed: Dec. 28, 2023

Related U.S. Application Data
(63) Continuation of application No. PCT/ES2022/070416, filed on Jun. 29, 2022.

(30) Foreign Application Priority Data
Jun. 30, 2021 (ES) P202130614

(51) Int. Cl. C12M 1/00; C12M 3/00

(52) U.S. Cl. CPC C12M 31/10; C12M 21/02; C12M 23/48; C12M 41/06

(57) ABSTRACT

A lighting device and incubator for microalgae growth experiments. The device includes at least one light source having light emitting diodes for illuminating microalgae, and a control unit configured to control the switching on and off of the light emitting diodes and to vary the light intensity of the light emitting diodes. The light source includes RGB LEDs, and each RGB LED has a housing that houses a red light emitting diode, a green light emitting diode, a blue light emitting diode and a microcontroller that communicates with the control unit to vary the light intensity of the red light emitting diode, the green light emitting diode and the blue light emitting diode of the RGB LED, such that the color and intensity of the light emitted by each RGB LED are modified by the control unit.

### CROSS-REFERENCE TO RELATED APPLICATIONS

This application relates to and claims the benefit and priority to International Application No. PCT/ES2022/070416, filed Jun. 29, 2022, which claims the benefit and priority to Spanish Application No. P202130614, filed Jun. 30, 2021, each of which is incorporated herein by reference in its entirety.

### FIELD

The present invention relates to a lighting device for performing small and medium scale microalgae growth experiments in research laboratories, as well as an incubator incorporating the lighting device for performing microalgae growth experiments.

### BACKGROUND

In recent times, microalgae research has gained a lot of importance due to the different uses that can be given to them. Microalgae, among other applications, can be used to produce biofuels, pharmaceuticals, food, to treat wastewater or industrial water, for air conditioning and insulation of buildings, etc.

Microalgae cannot be harvested in large quantities directly from nature and must therefore be cultivated. For the different uses of microalgae to be economically viable, the production costs of microalgae need to be significantly reduced.

Currently, large-scale artificial cultivation of microalgae can be classified into open systems, mainly large ponds where microalgae are grown in the open air, and closed systems, where microalgae are grown inside photobioreactors, thus achieving a higher quality product. Regardless of the system employed, light is an essential factor for microalgae growth, and varies significantly with time of exposure to light, light intensity, and light spectrum. In open systems the light used is natural, and the low production rates are compensated by the large volumes of microalgae grown in the ponds, but in photobioreactors, which have a significantly smaller volume, an adequate artificial light source is required for microalgae growth.

The light demand required by microalgae for proper growth is much more complex than the light required by other types of plants, and varies significantly with each species of microalgae. For this reason, it is necessary to carry out small-scale experiments in research laboratories to determine the optimal conditions for the growth of each species of microalgae, so that these conditions can later be reproduced in photobioreactors on an industrial scale. Experiments are generally carried out with white light fluorescent lamps, and in some cases colored filters are used to modify the color of the light from the fluorescent lamps.

CN201746536U discloses a lighting device for performing microalgae growth experiments comprising a light source having light emitting diodes (leds) for illuminating the microalgae and a control unit configured to control the switching on and off of the leds and to vary the light intensity of the leds. The lighting device is arranged in a temperature-controlled microalgae incubator in which the microalgae are placed. The incubator has an upper shelf with two led light sources and a lower shelf with fluorescent lamps, so that the incubator allows the difference in growth of microalgae illuminated with leds to be tested against microalgae illuminated with conventional fluorescent lamps. The light source comprises a panel with 100 high-brightness leds in red, green, blue, yellow and white, and the control unit varies the on/off and light intensity of the leds to simulate the gradual change of sunlight.

### SUMMARY

Provided are a lighting device and an incubator for performing microalgae growth experiments.

An aspect of the invention relates to a lighting device for performing microalgae growth experiments comprising at least one light source having leds for illuminating the microalgae and a control unit configured to control the switching on and off of the leds and to vary the light intensity of the leds. The light source comprises RGB LEDs, each RGB LED has a housing that houses a red led, a green led and a blue led and a microcontroller that is connected to the control unit to vary the light intensity of the red led, the green led and the blue led of the RGB LED, such that the color and intensity of the light emitted by each RGB LED are modified by the control unit.

Another aspect of the invention relates to an incubator for performing microalgae growth experiments comprising shelves on which containers with the microalgae are placeable, at least one light source arranged on each shelf having leds to illuminate the microalgae, and a control unit configured to control the switching on and off of the leds and to vary the light intensity of the leds. The light source comprises RGB LEDs, each RGB LED has a housing that houses a red led, a green led and a blue led, and a microcontroller that is connected to the control unit to vary the light intensity of the red led, the green led and the blue led of the RGB LED, such that the color and intensity of the light emitted by each RGB LED are modified by the control unit.

The control unit allows each RGB LED to be controlled independently from the light source, so that the switch-on time, the light intensity and the type of color of each RGB LED can be modified. The RGB LEDs combine the primary colors red, green and blue to obtain any color, which allows the microalgae to be illuminated with the required color and intensity and for the required time, and therefore an infinite number of experiments can be carried out that can later be scaled up to an industrial level to cultivate microalgae in photobioreactors. For example, CN201746536U uses individual five-color leds, so it cannot change the color at the led level, which limits the experiments to the colors of the leds. Also, by using single-color led, if a single color wanted to be used, the leds of the other colors must be kept off, so CN201746536U uses high-brightness leds to achieve the required light intensity for microalgae growth.

These and other advantages and features of the invention will become apparent in view of the figures and the detailed description of the invention.

### BRIEF DESCRIPTION OF THE DRAWINGS

FIG. 1 shows a perspective view of a light source of a lighting device according to an example.

FIG. 2 shows an exploded perspective view of the light source of FIG. 1.

FIG. 3 shows a schematic example of the electrical and data connections of the light source in FIG. 1.

FIG. 4 shows an incubator for microalgae growth experiments with light sources as shown in FIG. 1.

FIG. 5 shows an example of an arrangement of the light sources on a shelf of the incubator of FIG. 4.

FIG. 6 shows another example of an arrangement of the light sources on a shelf of the incubator of FIG. 4.

FIG. 7 shows a schematic example of the electrical and data connections of the light sources of the incubator of FIG. 4.

FIG. 8 shows the phycobiliprotein and biomass production of a cyanobacterial strain exposed to different colors with the lighting device.

### DETAILED DESCRIPTION

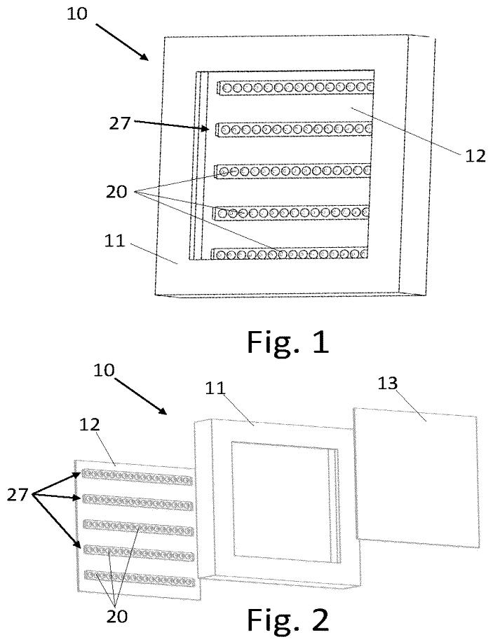

The invention relates to a lighting device for performing microalgae growth experiments comprising at least one light source 10 having leds 20 for illuminating the microalgae and a control unit 30 configured to control the switching on and off of the leds 20 and to vary the light intensity of the leds 20. See FIGS. 1 to 3.

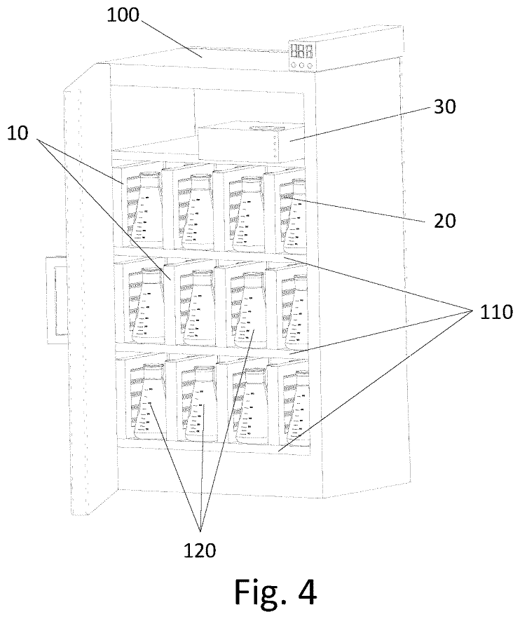

The lighting device is used to illuminate microalgae strains in a controlled environment for small-scale experiments to optimize the production of the microalgae strain or its products of interest. The lighting device can be installed in temperature-controlled rooms, incubators (as shown in FIG. 4) or any other controlled environment that allows microalgae growth.

The light source 10 comprises RGB LEDs (light emitting diodes) 20, wherein each RGB LED 20 has a housing that houses a red 21, green 22 and blue 23 led and a microcontroller 24 that is connected to the control unit 30 to vary the light intensity of the red 21, green 22 and blue 23 leds of the RGB LED 20, such that the color and intensity of the light emitted by each RGB LED 20 are modified by the control unit 30.

In this way, the lighting device has as many microcontrollers 24 as there are RGB LEDs 20 in the light source 10, so that the control unit 30 can independently select for each RGB LED 20 the combination of red, green and blue colors to obtain the required color, the light intensity of the RGB LED 20, and the switch-on time.

The lighting device has a power supply to electrically power the RGB LEDs 20 of the light source 10 and the control unit 30. The lighting device may have its own power supply, or a transformer and a conventional plug to connect to the grid. Preferably, the control unit 30 has the power supply integrated.

FIGS. 1 and 2 show an example of the light source 10. The light source 10 has a frame 11 supporting a rear cover 12 and a front diffuser 13, the rear cover 12 has a front face on which the RGB LEDs 20 are arranged and the front diffuser 13 is arranged on the front face of the rear cover 12 to homogenize the light emitted by the RGB LEDs 20.

The frame 11 defines a housing in which the rear cover 12 with the RGB LEDs 20 and the front diffuser 13 are housed, providing rigidity to the light source 10 for its correct transport, fixing and use. The rear cover 12 that supports the RGB LEDs 20 is made of an opaque material to direct the light towards the front diffuser 13. The front diffuser 13 is made of a translucent material that allows the light to pass through, homogenizing it and directing it towards the microalgae.

As can be seen in the figures, the RGB LEDs 20 are arranged in led strips 27 on the front face of the rear cover 12. Alternatively, the rear cover 12 with the RGB LEDs may be a single element, such as an RGB LED panel, or an RGB LED panel that is arranged on the rear cover 12.

Preferably, the led strips 27 extend parallel to each other and are arranged horizontally on the rear cover 12, so that a homogeneous and compact distribution of the RGB LEDs 20 is obtained, allowing the light to be concentrated.

Preferably, the lighting device further comprises an atmospheric sensor 40 for measuring the pressure, temperature and humidity of the environment in which the lighting device is arranged. The atmospheric sensor 40 is employed by the control unit 30 to monitor the environment in which the microalgae cultivation experiment is performed and to check that the pressure, temperature and humidity conditions are in accordance with the experiment. The atmospheric sensor 40 is a single device that integrates a pressure sensor, a temperature sensor and a humidity sensor. Alternatively, separate sensors can be used to measure the pressure, temperature and humidity of the environment.

Preferably, the lighting device further comprises at least one light sensor 50 for measuring the light intensity of the environment in which the lighting device is arranged. The light sensor 50 may be a photoresistor or LDR having a resistance that varies as a function of the light incident on the sensor. The light sensor 50 is employed by the control unit 30 to check that the light source 10 is correctly emitting light, and the control unit 30 may have an alarm management system to alert the user when the light source 10 is not illuminated according to the required light intensity.

Even more preferably, the lighting device further comprises a wireless data transmitter and receiver 60 that is associated with an external electronic device 70 for sending and receiving data of the status and switching on of each RGB LED 20, data of the light intensity and color of each RGB LED 20, and for sending to the external electronic device 70 data of the light intensity, temperature, humidity, and pressure of the environment in which the lighting device is arranged. The wireless data transmitter and receiver 60 may be a modem with 4G connectivity.

The external electronic device 70 may be a computer, smartphone, tablet or the like with a custom-designed software application, through which the lighting device can be remotely controlled. The software application allows the parameterization of the light intensity and color of each RGB LED 20, and allows programming the switch-on time of the light source 10, as well as the display of variables such as the temperature, pressure and humidity measured by the atmospheric sensor 40, or the light intensity measured by the light sensor 50.

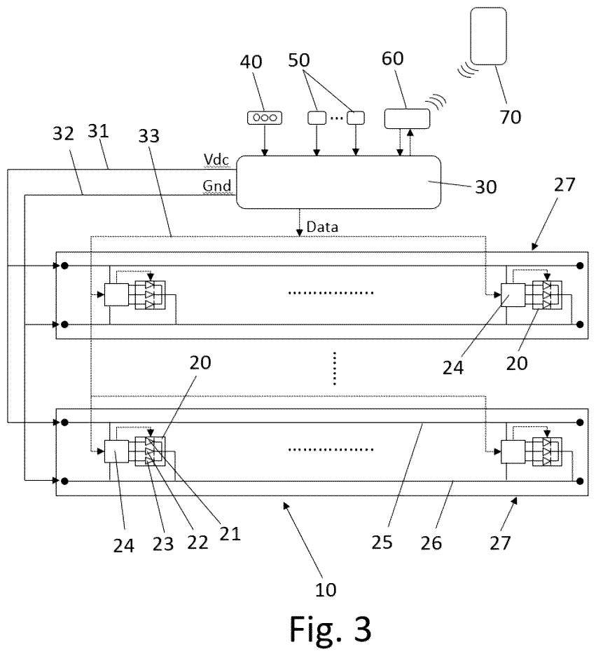

FIG. 3 shows a non-limiting schematic example of the electrical and data connections of a lighting device as shown in FIGS. 1 and 2 with several led strips 27 of RGB LEDs 20. Each led strip 27 has several RGB LEDs 20, and each RGB LED 20 has a housing that houses a red led 21, a green led 22 and a blue led 23 and a microcontroller 24 that is connected to the control unit 30 to vary the light intensity of the red led 21, the green led 22 and the blue led 23 of the RGB LED 20. Each led strip 27 has power cables 25 and 26 which extend longitudinally along the led strip 27 and which are electrically connected to power cables 31 and 32 of the control unit 30. The control unit 30 has a data bus 33 represented with a dotted line which is connected to the microcontroller 24 of each RGB led 20 to control the timing and switching on of the RGB LEDs 20, their light intensity and color type. The control unit 30 is operatively connected with the atmospheric sensor 40, the light sensors 50 and the wireless data transmitter and receiver 60 which is associated with the external electronic device 70.

The lighting device described above may comprise several light sources 10 controlled by the control unit 30. For example, the light sources may be arranged in a temperature-controlled room where there is a microalgae culture, or they may be arranged in a laboratory incubator as depicted in FIGS. 4 to 7.

Accordingly, the invention also relates to an incubator 100 for performing microalgae growth experiments comprising shelves 110 on which containers 120 with the microalgae are placeable, at least one light source 10 arranged on each shelf 110 having leds 20 to illuminate the microalgae, and a control unit 30 configured to control the switching on and off of the leds 20 and to vary the light intensity of the leds 20. The light source 10 comprises RGB LEDs 20, wherein each RGB LED 20 has a housing that houses a red led 21, a green led 22 and blue led 23 and a microcontroller 24 that is connected to the control unit 30 for varying the light intensity of the red led 21, the green led 22 and the blue led 23 of the RGB LED 20, such that the color and intensity of the light emitted by each RGB LED 20 are modified by the control unit 30.

Preferably, on each shelf 110 are arranged lines 130 of containers 120, each line 130 having a left side and a right side, and each shelf 110 has rows 140 of light sources 10, with at least one light source 10 per row 140, and at least one side of each line 130 of containers 110 is illuminated by a respective row 140 of light sources 10. This ensures that all containers 120 on each shelf 110 are directly illuminated and that there are no containers 120 shading others.

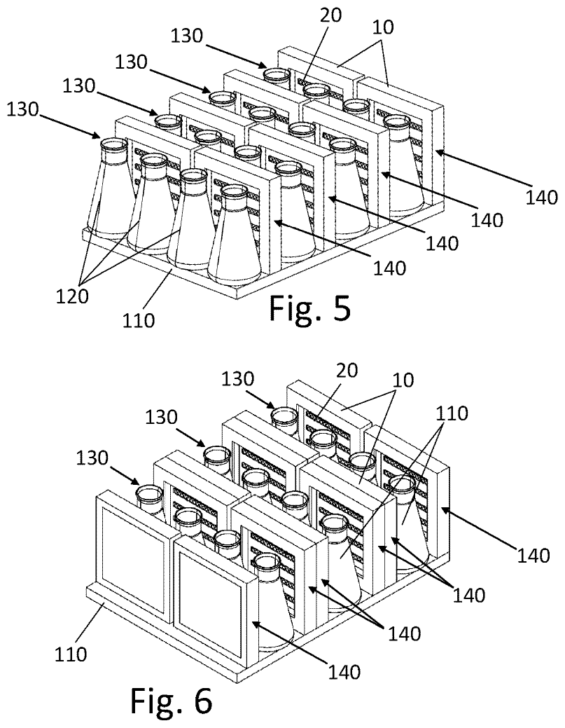

As shown in FIG. 5, only one side of each line 130 of containers 110 is illuminated by a respective row 140 of light sources 10. Alternatively, and preferably, as shown in FIG. 6, both left and right sides of each line 130 of containers 120 are illuminated by a respective row 140 of light sources 10. Some types of microalgae have a density that makes it difficult for light to pass through them, so rather than employing high brightness light sources 10 that can illuminate from only one side, it is preferable to employ light sources 10 that are lower in intensity but illuminate the containers 120 from both sides. In containers 120 that have a volume of 250 ml there is no problem for light to pass through, however, illuminating from both sides is useful when you want to increase the amount of light the crop receives to improve growth.

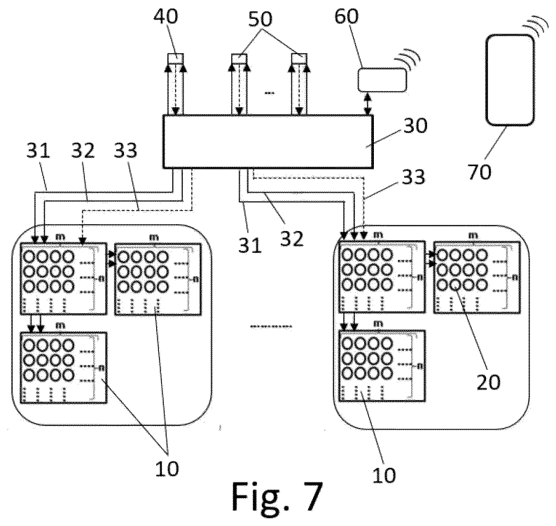

FIG. 7 shows a non-limiting example of a lighting device as shown in FIGS. 1 and 2 with several led strips 27 of RGB LEDs 20. Each led strip 27 has several RGB LEDs 20, and each RGB LED 20 has a housing which houses a red led 21, a green led 22 and a blue led 23, and a microcontroller 24 that is connected to the control unit 30 for varying the light intensity of the red led 21, the green led 22 and the blue led 23 of the RGB LED 20. Each led strip 27 has power cables 25 and 26 extending longitudinally along the led strip 27, which are electrically connected to power cables 31 and 32 of the control unit 30. The control unit 30 has a data bus 33 represented with a dotted line which is connected to the microcontroller 24 of each RGB LED 20 to control the timing and switching on of the RGB LEDs 20, their light intensity and color type. The control unit 30 is operatively connected with the atmospheric sensor 40, the light sensors 50 and the wireless data transmitter and receiver 60 which is associated with the external electronic device 70.

FIG. 7 shows a non-limiting schematic example of the electrical and data connections of the power supplies 10 arranged on the shelves 110 of an incubator 100 as shown in FIG. 4. The electrical and data connections in FIG. 7 are identical to those depicted in FIG. 3, but FIG. 7 depicts the connection of several light sources 10.

Each light source 10 of the incubator has several RGB LEDs 20, and each RGB LED 20 has a housing that houses a red led 21, a green led 22 and a blue led 23, and a microcontroller 24 that is connected to the control unit 30 to vary the light intensity of the red led 21, the green led 22 and the blue led 23 of the RGB LED 20. The control unit 30 has power cables 31 and 32 for powering the light sources and a data bus 33 which is connected to the microcontroller 24 of each RGB LED 20 to control the timing and switching on of the RGB LEDs 20, their light intensity and color type.

The incubator 100 comprises an atmospheric sensor 40 for measuring the pressure, temperature and humidity inside the incubator where the microalgae are available, and the control unit 30 is operatively connected with the atmospheric sensor 40.

The incubator 100 has a light sensor 50 on each shelf 110 to measure the intensity of each shelf 110 of the incubator, and the control unit 30 is operatively connected with the light sensors 50 to check that the light sources 10 on each shelf 110 are operating within the parameters required by the experiment.

The incubator further comprises a wireless data transmitter and receiver 60 that is associated with an external electronic device 70 for sending and receiving data of the status and power on of each RGB LED 20, data of the light intensity and color of each RGB LED 20, and for sending to the external electronic device 70 data of the light intensity, temperature, humidity, and pressure of the incubator in which the microalgae are available.

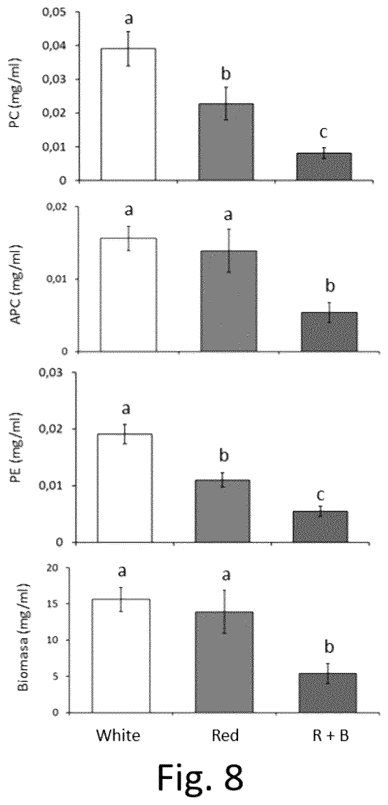

FIG. 8 shows an experiment with the lighting device, where cyanobacteria were exposed to white light, red light and a combination of red and blue light. To obtain the white light, all the RGB 20 LEDs of the light source 10 were switched on in white light, to obtain the red light, all the RGB 20 LEDs of the light source 10 were switched on in red light, and to obtain the combined light, some of the RGB 20 LEDs were switched on in red light and some of the RGB 20 LEDs were switched on in blue light.

Specifically, the phycobiliproteins of a cyanobacterial strain (phycocyanin PC, allophycocyanin APC and phycoerythrin PE), as well as the biomass of the cyanobacterial strain, were exposed to white, red and the combination of red and blue light. As can be seen in the figure, the color used has a great influence on relevant parameters in terms of microalgae production. It can be seen how white and red light achieved the best pigment and biomass production values in the strain studied. Accordingly, given these results, when cultivating this strain for industrial exploitation, the color of the led source chosen for the photobioreactor would be red due to its lower energy consumption and its good results when producing the products of interest.

In following clauses additional embodiments are disclosed.

Clause 1. Lighting device for performing microalgae growth experiments, comprising at least one light source (10) having leds (20) for illuminating the microalgae and a control unit (30) configured to control the switching on and off of the leds (20) and to vary the light intensity of the leds (20), the light source (10) comprises RGB LEDs (20), and in that each RGB led (20) has a housing that houses a red led (21), a green led (22) and a blue led (23), and a microcontroller (24) that is connected to the control unit (30) to vary the light intensity of the red led (21), the green led (22) and the blue led (23) of the RGB LED (20), such that the color and intensity of the light emitted by each RGB led (20) are modified by the control unit (30).

Clause 2. Lighting device according to clause 1, wherein the light source (10) has a frame (11) supporting a rear cover (12) and a front diffuser (13), the rear cover (12) has a front face on which the RGB LEDs (20) are arranged and the front diffuser (13) is arranged on the front face of the rear cover (12) to homogenize the light emitted by the RGB LEDs (20).

Clause 3. Lighting device according to clause 2, wherein the RGB LEDs (20) are arranged in led strips (27) on the front face of the rear cover (12).

Clause 4. Lighting device according to the preceding clause, wherein the led strips (27) extend parallel to each other and are arranged horizontally on the rear cover (12).

Clause 5. Lighting device according to any one of clauses 1 to 4, further comprising an atmospheric sensor (40) for measuring the pressure, temperature and humidity of the environment in which the lighting device is arranged.

Clause 6. Lighting device according to any one of the preceding clauses, further comprising at least one light sensor (50) for measuring the light intensity of the environment in which the lighting device is arranged.

Clause 7. Lighting device according to the preceding clause, further comprising a wireless data transmitter and receiver (60) that is associated with an external electronic device (70) for sending and receiving data of the status and switching on of each RGB LED (20), data of the light intensity and color of each RGB LED (20), and for sending to the external electronic device (70) data of the light intensity, temperature, humidity, and pressure of the environment in which the lighting device is arranged.

Clause 8. Incubator for performing microalgae growth experiments, comprising shelves (110) on which containers (120) with the microalgae are placeable, at least one light source (10) arranged on each shelf (110) having leds (20) to illuminate the microalgae, and a control unit (30) configured to control the switching on and off of the leds (20) and to vary the light intensity of the leds (20), the light source (10) comprises RGB LEDs (20), and in that each RGB LED (20) has a housing that houses a red led (21), a green led (22) and a blue led (23), and a microcontroller (24) that is connected to the control unit (30) to vary the light intensity of the red led (21), the green led (22) and the blue led (23) of the RGB LED (20), such that the color and intensity of the light emitted by each RGB LED (20) are modified by the control unit (30).

Clause 9. Incubator according to the preceding clause, wherein the light source (10) has a frame (11) supporting a rear cover (12) and a front diffuser (13), the rear cover (12) has a front face on which the RGB LEDs (20) are arranged and the front diffuser (13) is arranged on the front face of the rear cover (12) to homogenize the light emitted by the RGB LEDs (20).

Clause 10. Incubator according to the preceding clause, wherein the RGB LEDs (20) are arranged in led strips (27) on the front face of the rear cover (12).

Clause 11. Incubator according to the preceding clause, wherein the led strips (27) extend parallel to each other and are arranged horizontally on the rear cover (12).

Clause 12. Incubator according to any one of clauses 8 to 11, further comprising an atmospheric sensor (40) for measuring the pressure, temperature and humidity inside the incubator where microalgae are placeable.

Clause 13. Incubator according to any one of clauses 8 to 12, further comprising a light sensor (50) on each shelf (110) for measuring the intensity of each shelf (110) of the incubator.

Clause 14. Incubator according to the preceding clause, further comprising a wireless data transmitter and receiver (60) which is associated with an external electronic device (70) for sending and receiving data of the status and switching on of each RGB LED (20), data of the light intensity and color of each RGB LED (20), and for sending to the external electronic device (70) data of the light intensity, temperature, humidity, and pressure of the incubator where the microalgae are placeable.

Clause 15. Incubator according to any one of clauses 8 to 14, wherein on each shelf (110) are arranged lines (130) of containers (120), each line (130) having a left side and a right side, and each shelf (110) has rows (140) of light sources (10), with at least one light source (10) for each row (140), and wherein at least one side of each row (130) of containers (120) is illuminated by a respective row (140) of light sources (10).

Clause 16. Incubator according to the preceding clause, wherein both left and right sides of each line (130) of containers (120) are illuminated by a respective row (140) of light sources (10).

What is claimed is:

1. A lighting device for performing a microalgae growth experiment, the lighting device comprising:
a plurality of RGB light emitting diodes that each includes a housing in which resides a red light emitting diode, a green light emitting diode, a blue light emitting diode, and a microcontroller electrically coupled to each of the red light emitting diode, the green light emitting diode and the blue light emitting diode; and
a control unit that communicates with the microcontroller of each of the plurality of RGB light emitting diodes to control the switch-on time and light intensity of the red light emitting diode, the green light emitting diode and the blue light emitting diode of each of the plurality of RGB light emitting diodes to obtain a desired color output of each of the plurality of RGB light emitting diodes.

2. The lighting device according to claim 1, further comprising a frame that supports a rear cover and a front diffuser, the rear cover having a front face on which the plurality of RGB light emitting diodes are arranged, and the front diffuser is arranged on the front face of the rear cover, the front diffuser being configured to homogenize light emitted by the plurality of RGB light emitting diodes.

3. The lighting device according to claim 2, wherein the plurality of RGB light emitting diodes are arranged in strips on the front face of the rear cover.

4. The lighting device according to claim 3, wherein the strips extend parallel to each other and are arranged horizontally on the rear cover.

5. The lighting device according to claim 1, further comprising one or more sensors that are configured to measure one or more of a pressure, a temperature, and a humidity of the environment in which the microalgae cultivation experiment is performed.

6. The lighting device according to claim 1, further comprising a light sensor that are configured to measure the light intensity of the environment in which the lighting device is arranged.

7. The lighting device according to claim 6, wherein the control unit is configured to employ the light sensor to determine if the plurality of RGB light emitting diodes are correctly emitting light.

8. The lighting device according to claim 7, wherein the control unit includes an alarm management system that is configured to initiate an alarm when one or more of the plurality of RGB light emitting diodes are not illuminated according to a required light intensity.

9. The lighting device according to claim 5, further comprising a light sensor that is configured to measure the light intensity of the environment in which the lighting device is arranged.

10. The lighting device according to claim 9, further comprising a wireless data transmitter and receiver that is configured to exchange data associated with one or more of the measured light intensity, pressure, temperature and humidity with an external electronic device.

11. An assembly for performing microalgae growth experiments, the assembly comprising;
a shelf configured to support a plurality of containers containing microalgae;
a light assembly that is configured to illuminate the microalgae when the plurality of containers are supported on the shelf, the light assembly including a plurality of lighting devices that each includes a plurality of RGB light emitting diodes, each of the plurality of lighting devices is arranged to direct light to at least one of the plurality of containers, each RGB light emitting diode including a housing in which resides a red light emitting diode, a green light emitting diode, a blue light emitting diode, and a microcontroller electrically coupled to each of the red light emitting diode, the green light emitting diode and the blue light emitting diode; and
a control unit that communicates with the microcontroller of each of the RGB light emitting diodes to control the switch-on time and light intensity of the red light emitting diode, green light emitting diode and blue light emitting diode of each of the plurality of RGB light emitting diodes to obtain a desired color output for each of the RGB light emitting diodes.

12. The assembly according to claim 11, wherein one or more of the plurality of lighting devices is supported on the shelf.

13. The lighting device according to claim 11, wherein each of the lighting devices includes a frame that supports a rear cover and a front diffuser, the rear cover having a front face on which the plurality of RGB light emitting diodes are arranged, and the front diffuser is arranged on the front face of the rear cover, the front diffuser being configured to homogenize light emitted by the plurality of RGB light emitting diodes.

14. The assembly according to claim 13, wherein the plurality of RGB light emitting diodes of each lighting device are arranged in strips on the front face of the rear cover.

15. The assembly according to claim 14, wherein the strips extend parallel to each other and are arranged horizontally on the rear cover.

16. The assembly according to claim 11, further comprising an atmospheric sensor for measuring the pressure, temperature, and humidity where the microalgae are placeable.

17. The assembly according to claim 11, further comprising a light sensor that is configured to measure a light intensity at the shelf.

18. The assembly according to claim 17, further comprising a wireless data transmitter and receiver which is associated with an external electronic device for sending and receiving data of the status and switching on of each of the plurality of RGB light emitting diodes, data of the light intensity and color of each of the plurality of RGB light emitting diodes, and for sending to the external electronic device data of the light intensity, temperature, humidity, and pressure where the microalgae are placeable.

19. The assembly according to claim 11, further comprising the plurality of containers being supported on the shelf in a plurality of rows, each container having a first side and a second side that faces in opposite directions, at least one of the plurality of lighting devices being arranged in each row facing the first side of at least one of the plurality of containers.

20. The assembly according to claim 19, further comprising at least one of another of the plurality of lighting devices being arranged facing the second side of the at least one of the plurality of containers such that the at least one of the plurality of containers is illuminated on both the first and second sides.
